(19) United States
(12) Patent Application Publication
(10) Pub. No.: US 2005/0197714 A1
(43) Pub. Date: Sep. 8, 2005

(54) System, system devices, and methods for regulating nutrient absorption and caloric intake

(76) Inventor: Peter H. Sayet, Fort Lauderdale, FL (US)

Correspondence Address: AKERMAN SENTERFITT, P.O. BOX 3188, WEST PALM BEACH, FL, 33402-3188, US

(21) Appl. No.: 10/791,179

(22) Filed: Mar. 2, 2004

(51) Int. Cl. A61F 2/04

(52) U.S. Cl. 623/23.65; 623/902

(57) ABSTRACT

A system (100) for regulating absorption and caloric intake can include an elongated tube (110) having a stomach portion (114) and a lower intestine portion (116) where the elongated tube defines a passage (114) to guide ingested material through the stomach (102) and through a portion of the small intestine (104). The system also includes a non-invasive stomach stricture device (150) having a clamping structure (152) for regulating the rate of flow of ingested material through the elongated tube where the clamping structure reduces the capacity of the passage by clamping a portion of the elongated tube through the exterior of the stomach.

### CROSS REFERENCE TO RELATED APPLICATIONS

Not Applicable.

### FIELD OF THE INVENTION

This invention relates to a system, system devices, and methods for regulating nutrient absorption and caloric intake of the user when used on the digestive system.

### BACKGROUND

Obesity is a great problem in the United States and throughout the world. Many solutions to this problem have involved the use of drugs to, for example, block fat absorption or suppress appetite. See U.S. Pat. No. 5,911,992. Another solution involves an apparatus adapted to be fitted within the mouth of a user so as to control the amount of solid food that the user may intake into his or her digestive system. See U.S. Pat. No. 4,471,771.

One surgical procedure for combating obesity is known as vertical banded gastroplasty (VBG). During the procedure, the surgeon incises a circular window through the stomach a few inches below the cardiac orifice of the esophagus, which is closed off using surgical staples around the perimeter. A surgical stapler is also used to further create a small pouch by attaching a row of staples leading from the window toward the esophagus. The pouch creates a restricted orifice and a reduced inside surface area within the stomach. A polypropylene band can be placed through the window, around the outlet of the pouch, and secured to the pouch via stitches. The band can control the size of the outlet and keeps it from stretching. As a result, the VBG limits the amount of food the patient can eat at one time and results in weight loss in most patients.

The problems associated with the VBG procedure, and other such similar surgical protocols (like the stomach bypass) which require some combination of stapling, surgical stitching and/or resectioning of the stomach and/or intestines are also well documented in the medical journals. During the ideal VBG procedure, the surgeon can carefully measure the pouch to ensure that it will hold approximately one ounce of solid food; however, such measurements can be inaccurate and even omitted by non-diligent surgeons. The variety of adverse complications associated with the invasive incising of the stomach and the placement of an elongated staple line down almost the entire vertical line (and around the circumference of the excised hole) of the stomach can include infection, gastric fluid leakage and staple line detachment. Further, approximately five percent of the patients who undergo these surgical procedures continue to overeat and inadvertently cause the staple line to rip open. Additionally, although it is possible in some cases to restore some portions of the stomach back to its original pre-surgical functions by additional surgery, it is unlikely that any patient receiving such surgical interventions will be capable of regaining a fully functional recovery from such surgery.

There are a number of other surgical protocols using an outside closure or invasive technique which have also been devised and described with the common primary objective of reducing the amount of usable inside stomach surface area. To date, all of the documented invasive surgical protocols share similar adverse events as previously described. Additionally, products have been designed to partially surround the stomach from the outside and, using a hydraulic bladder system, constrict the opening of the cardiac orifice between the lower esophagus and upper stomach in such a way as to restrict the amount of food that can be ingested, in a similar objective as the previously described invasive surgical protocols. Nevertheless, such devices can slip from their intended position and cause constriction at a point higher and/or lower on the esophagus and imbed the device in the surrounding tissue, causing further complications. Consequently, there is still a need for a product or procedure to effectively treat obesity.

### SUMMARY OF THE INVENTION

The present invention avoids and overcomes certain of the above-mentioned problems and shortcomings. The invention provides a system having at least two devices that cooperatively operate to regulate nutrient absorption and caloric intake of a user. Accordingly, the system provides an enhanced regulation of nutrient absorption and caloric intake compared to use of single devices; however, the individual components of the system can also be used individually to regulate nutrient absorption and caloric intake of a user. Also, it should be noted that implementation of the system does not produce any permanent changes to the anatomy of the user's stomach or intestines, such as the irreversible consequences associated with VBG and other stomach bypass procedures, and therefore, the system, and its individual components, can be removed to allow the patient to return to the normal digestive process.

In one aspect of the invention, a system for regulating nutrient absorption and caloric intake is provided. The system can include an elongated tube positioned on the inside of the stomach having a stomach portion and a lower intestine portion where the elongated tube can define a passage to guide ingested material through the stomach and through a portion of the small intestine. The system can also include a non-invasive stomach stricture device positioned on the outside of the stomach having a positing member where the positioning member positions the elongated tube within the stomach. The non-invasive stomach stricture device can also include a clamping structure for regulating the rate of flow of ingested material through the elongated tube where the clamping structure alters the capacity of the portion of stomach through which the elongated tube passes. The elongated tube can also include an inflatable member.

In one embodiment, the clamping structure can prevent the elongated tube from passing out of the stomach. Also, the positioning member can position the clamping structure on the stomach and relative to the elongated tube. The clamping structure can include a front side member and a backside member, and a connector for coupling to the front side member and the backside member. Also, the positioning member can be dimensioned to clamp the elongated tube through the stomach and reduce the capacity of the passage. Accordingly, the elongated tube can include a collar designed to be clamped by the positioning member and the positioning member can be dimensioned to clamp the collar for suspending a portion of the elongated tube within the stomach.

In another embodiment, the positioning member can prevent the elongated tube from passing out of the stomach. Also, the non-invasive stomach stricture device can include a clamp adjusting mechanism. The clamp adjusting mechanism can include an actuator for operating the clamp adjusting mechanism.

In an other aspect of the invention, a system for regulating nutrient absorption and caloric intake is provided. The system can include an elongated tube having a stomach portion and a lower intestine portion. The elongated tube can define a passage to guide ingested material through the stomach and through a portion of the small intestine and a non-invasive stomach stricture device having a clamping structure can be provided. The clamping structure can prevent the elongated tube from exiting the stomach.

In one embodiment, the non-invasive stomach stricture device can include a positioning member for positioning the elongated tube within the stomach. The arrangement of the clamping structure can regulate the rate of flow of ingested material through the elongated tube. Also, the arrangement of the clamping structure can alter the capacity of the portion of stomach through which the elongated tube passes.

In another embodiment, the elongated tube can include an inflatable member. The clamping structure can also include a front side member, a backside member, and a connector for coupling to the front side member and the backside member. The front side member, the backside member, and the connector can be integral. Also, the elongated tube can include a collar designed to be clamped by the positioning member and the positioning member can be dimensioned to clamp the collar for suspending a portion of the elongated tube within the stomach. The non-invasive stomach stricture device can include a clamp adjusting mechanism. Also, the clamp adjusting mechanism can include an actuator for operating the clamp adjusting mechanism.

In another aspect of the invention, a non-invasive stomach stricture device is provided having a front side member, a backside member, and a connector for coupling the front side member to the backside member to form a clamp where the clamp can be designed to clamp a stomach. The clamp can be designed to have a size and shape suitable for fitting adjacent to the trachea side of the upper quadrant of a stomach for regulating nutrient absorption and caloric intake by reducing the capacity of the stomach. Further, the backside member and the front side member can include stomach contacting surfaces and the stomach contacting surfaces can be free of piercing projections. Also, one or more of the stomach contacting surfaces can include structure for permitting fluid flow.

In another arrangement, a positioning member can be provided for coupling to the front side member and the backside member to form a clamping assembly. The front side member, connector, and backside member can be integral. Also, the backside member and the connector can be integral. The connector can include a slot adapted to receive the front side member so that the front side member can be coupled to the backside member to form a clamp. The backside member and the front side member can each include an anchoring slot where each anchoring slot can be adapted to receive an end of the positioning member. Also, each slot and the positioning member can include corresponding engagement structure for coupling the positioning member to the front side member and the backside member. The front side member and the backside member can also include an aperture through which the front side member and the backside member can be sutured to the stomach.

In another aspect of the invention, a non-invasive stomach stricture device is provided. The non-invasive stomach stricture device can include a front side member, a backside member, and a connector connecting the front side member to the backside member. The connector can be dimensioned to space the front side member from the backside member such that the device can be positioned over the stomach of a patient with the front side member over a front side of the stomach and the backside member positioned over the backside of the stomach, and the stomach will be constricted between the front side member and the backside member.

In one embodiment, the connector and the backside member can be integral. The connector can determine the distance between the front side member and the backside member. A positioning member can also be provided for positioning the stomach stricture device on the stomach of a patient. The positioning member can couple to a portion of the front side member and to a portion of the backside member. The positioning member can be adjustable relative to the front side member and the backside member. Also, the backside member and the front side member can each include an anchoring slot for receiving an end of the positioning member.

In another embodiment, the positioning member can include engagement structure for connecting with at least one corresponding engagement structure in at least one of the front side member and the backside member. The front side member can be separable from the backside member for allowing the device to be removed from a patient. Also, the device can be dimensioned for positioning adjacent to the trachea side of the upper quadrant of a human stomach to limit the rate of flow of ingested material into the stomach and to limit the digestion and absorption of the ingested material. The device can also be dimensioned for positioning adjacent to the upper quadrant of a human stomach, substantially perpendicular to the esophagus, to limit the rate of flow of ingested material into the stomach and to limit the digestion and absorption of the ingested material.

In another aspect of the inventive arrangements, a method for implanting a non-invasive stomach stricture device is provided. The method can include the step of providing a non-invasive stomach stricture device having a front side member, a backside member, and a connector for coupling the front side member to the backside member to form a clamp, where the clamp is designed to clamp a stomach. The method can also include coupling the backside member to the connector, positioning the backside member, coupled to the connector, adjacent to the trachea side of the upper quadrant of the human stomach. The method can further include coupling the front side member to the connector to form a clamp which constricts the stomach to limit the rate of flow of ingested material into the stomach and to limit the amount of usable inside surface area of the stomach.

In one arrangement, the step of placing the backside member adjacent to the trachea side of the upper quadrant of the human stomach can include excising a canal through fatty tissue on the backside of the stomach. The method can also include the step of connecting a connector to the front side member and the backside member.

In another aspect of the inventive arrangements, a device for regulating nutrient absorption and caloric intake is provided. The device can include an elongated tube having a stomach portion and a lower intestine portion where the elongated tube defines a passage to guide ingested material through the stomach and through a portion of the small intestine. The device can also include anchoring structure for anchoring the elongated tube to adjacent tissue. The stomach portion can include an esophageal portion for positioning substantially at the region where the esophagus meets the stomach and the esophageal portion can include the anchoring structure.

In one arrangement, the anchoring structure can include a cardiac orifice structure for anchoring a portion of the elongated tube to the cardiac orifice. The anchoring structure can include at least one of a collar, a web, and a reinforced portion. The device can also include anchoring structure at the lower intestine portion for anchoring the lower intestine portion to adjacent tissue of the patient. The anchoring structure can include at least one of a collar and a web for suturing.

In another arrangement, the stomach portion can include at least one opening. The device can also include structure for positioning the tube in the stomach of a patient. The positioning structure can include at least one inflation member. Further, at least four inflation members spaced substantially equidistantly about a circumference of the stomach portion can be provided. The device can also include a plurality of inlet openings located substantially at the region between the stomach portion and the lower intestine portion where the inlet openings can be elongated slots. The device can also include a plurality of exit openings located substantially at the region between anchoring structure an the stomach portion.

In another aspect of the inventive arrangements, a method for regulating nutrient absorption and caloric intake is provided. The method can include providing a device for regulating nutrient absorption and caloric intake having an elongated tube with a stomach portion and a lower intestine portion where the elongated tube can define a passage to guide ingested material through the stomach and through a portion of the small intestine, and an anchoring structure at the stomach portion for anchoring the elongated tube before the elongated tube enters the stomach. The method can also include positioning the device in a patient with the stomach portion substantially in the stomach of the patient and the lower intestine portion substantially in the lower intestine of the patient and securing the device in position in the patient. With such a method, a portion of the ingested material will pass through the passage without being digested by the patient.

In one arrangement, the method can also include the step of adjusting the length of the lower intestine portion where a longer length prevents absorption of ingested material through the lower intestine. Further, the method can include the step of providing inflation members secured to the stomach portion, and filling the inflation members after the stomach portion has been positioned in the stomach of the patient. Also, the method can include the step of securing the lower intestine portion to the lower intestine of the patient.

In another aspect of the inventive arrangements, a method of regulating nutrient absorption and caloric intake is provided. The method can include the steps of non-invasively reducing the capacity of the stomach and limiting the interaction of digestive agents with ingested substances. Further, reducing the capacity of the stomach includes clamping a portion of the stomach. The method can also include the step of selectively adjusting the clamping force. The limiting step can include funneling ingested material through a portion of the digestive system.

The above features and advantages of the present invention will be better understood with reference to the following figures and detailed description. It should be appreciated that the particular devices and methods illustrating the present invention are exemplary only and not to be regarded as limitations of the present invention.

### BRIEF DESCRIPTION OF THE DRAWINGS

There are presently shown in the drawings embodiments which are presently preferred, it being understood, however, that the invention is not limited to the precise arrangements and instrumentalities shown.

FIG. 1 is a schematic view of a system for regulating nutrient absorption and caloric intake illustrated in operation with a stomach in accordance with the inventive arrangements.

FIG. 2 is a perspective view of a non-invasive stomach stricture device illustrated in operation in accordance with the inventive arrangements.

FIG. 3 is an exploded view of a dissembled non-invasive stomach stricture device in accordance with the inventive arrangements.

FIG. 4 is a schematic of one embodiment of the clamp adjusting mechanism.

FIG. 5 is a cross-sectional view illustrating the connection of the front side member to the backside member taken along line 5-5 in FIG. 4.

FIG. 6A is a cross-sectional view of another embodiment of a non-invasive stomach stricture device in accordance with the inventive arrangements.

FIG. 6B is a partial cross-sectional view of the embodiment of FIG. 6A.

FIG. 6C is a partial cross-sectional view of the embodiment of FIG. 6B.

FIG. 7 is a schematic view of a device for regulating nutrient absorption and caloric intake in accordance with the inventive arrangements.

FIG. 8 is a schematic view of one embodiment of a device for regulating nutrient absorption and caloric intake in accordance with the inventive arrangements.

FIG. 9 is a schematic view of another embodiment of a device for regulating nutrient absorption and caloric intake in accordance with the inventive arrangements.

FIG. 10 is a cross-sectional view of another embodiment of a non-invasive stomach stricture device in accordance with the inventive arrangements.

### DETAILED DESCRIPTION

The present invention provides a system having at least two devices that synergistically operate to regulate nutrient absorption and caloric intake of a user. With the synergism, the system provides an enhanced regulation of nutrient absorption and caloric intake compared to use of single devices. Nevertheless, the individual components of the system can also be used individually to regulate nutrient absorption and caloric intake of a user. Also, it should be noted that implementation of the system does not produce any permanent changes to the anatomy of the user's stomach or intestines, such as the irreversible consequences associated with VBG and other stomach bypass procedures, and therefore, the system, and its individual components, can be removed to allow the patient to return to the normal digestive process. Further, as no permanent changes to anatomy are produced, the system can be dynamically adjusted to correspond to changing dietary needs.

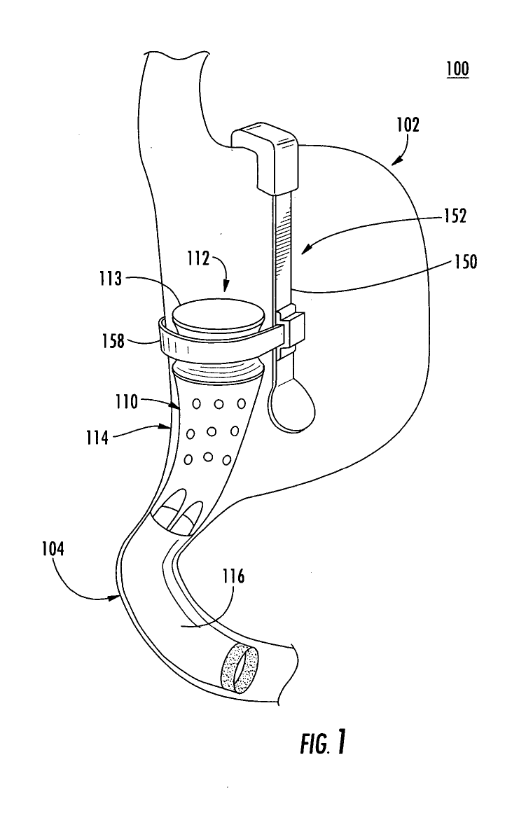

In accordance with the inventive arrangements, FIG. 1 illustrates a system 100 for regulating nutrient absorption and caloric intake that is arranged about stomach 102 and a portion of the small intestine 104. The system 100 can include an elongated tube positioned inside the stomach 110 defining a passage 112 and a non-invasive stricture device 150 having a clamping structure 152 and a positioning member 158. The elongated tube 110 can include a stomach portion 114 which can be positioned generally in the stomach 102 and a lower intestine portion 116 that can be positioned generally in a portion of the small intestine 104. Thus, the passage 112 provided by the elongated tube 110 can guide ingested material through the stomach 102 and through a portion of the small intestine 104.

The elongated tube 110 is shown as suspended within stomach 102. In this arrangement, the elongated tube 110 includes a collar 113 which can provide an opening to passage 112. The collar 113 can be designed to be clamped by the positioning member 158 so that the collar 113 is suspended within the stomach and the elongated tube 110 is positioned in the appropriate portion of the stomach. The dimensions and curvature of the collar 113 can correspond to the dimensions and curvature of the positioning member 158 so that the positioning member 158 securely suspends the elongated tube 110. The collar 113 can be constructed of a semi-rigid plastic and/or any appropriate biomaterial that can withstand the clamping forces produced by the positioning member 158, which can be constructed of a semi-rigid material, a flexible material, or an elastic material that can secure the collar 113. It should also be noted that the collar 113 can also be constructed of a suitably flexible material, such as flexible plastic, that can be compressed by the positioning member 158 and allow for deformation during patient movements to not restrict any range of patient movement.

The non-invasive stricture device 150 can also section the stomach by clamping the outside wall of the stomach, such that the inside wall is sectioned 102 into at least two portions where one portion receives food for normal digestion and nutrient absorption. This portion is the portion of the stomach in which the elongated tube can pass. The other portion of the stomach 102 is prevented by the clamping structure 152 from receiving food, and consequently prevents this portion of the stomach 102 from participating substantially in the digestive process. The size and capacity of the portion used for digestion can depend on the arrangement of the clamping structure 152. The arrangement of the clamping structure 152 can include the amount of clamping force provided, the positioning of the clamping structure 152, and/or the size of the clamping structure 152. By altering the arrangement, the clamping structure 152 can regulate the rate of flow of ingested material and alter the capacity of the portion of the stomach used for digestive purposes.

Accordingly, the clamping structure 152 can alter the capacity of the portion of stomach 102 through which the elongated tube 110 passes. The capacity can vary depending upon the shape, size, and placement of the clamping structure 152. Further, the capacity can also be varied over time to meet changing dietary needs by adjusting the position of the clamping structure 152 or by using one of the various adjustment mechanisms discussed below.

In an additional embodiment (not shown), the clamping structure 152 can reduce the capacity of the passage 112 by clamping a portion of the elongated tube 110 through the exterior of the stomach which results in the closing off of the inside wall of the stomach around the elongated tube at the designed positions that help position it. Accordingly, while the clamping structure 152 reduces the capacity of the passage 112, the capacity of the stomach 102 can also be reduced. The regulation of nutrient absorption and caloric intake can depend on the capacity of the passage 112 and the stomach 102 with greater reduction in capacity resulting in a greater reduction in nutrient absorption and caloric intake.

As used herein, capacity can relate to the amount of available volume through which ingested material can pass and/or can be filled; however, capacity can also relate to the rate at which the ingested material can pass and/or can be filled. Additionally, capacity can also relate to the amount and/or rate at which ingested material can be digested and/or absorbed. Accordingly, a reduction in capacity can be a reduction in the available volume through which ingested material can pass and/or can be filled, a reduction in the rate at which the ingested material can pass and/or can be filled, a reduction in the amount and/or rate at which ingested material can be digested and/or absorbed, or a combination thereof.

The elongated tube 110 can regulate the capacity in a variety of manners. For instance, the stomach portion 114 of the elongated tube 110 can include one or more openings 111 for permitting ingested material to exit and re-enter the stomach portion 114. Also, the openings 111 can permit digestive agents, such as stomach acid, to enter and exit the stomach portion 114. A plurality of openings 111 can allow for uniform digestion and nutrient absorption. These openings 111 can be a variety of sizes with the size of the opening also controlling the level of overall digestion.

Although the non-invasive stricture device 150 can regulate the capacity of the stomach 102 and/or the passage 112, the positioning member 158, in combination with the clamping structure 152 or individually, can also prevent the elongated tube 110 from passing out of the stomach 102. In the clamped arrangement, the positioning member 158 can prevent normal peristaltic functioning of the digestive system from passing the elongated tube 110 along the normal course of the digestive tract of out of the stomach 102 and along the small intestine 104. Additionally, the positioning member 158 can also prevent the elongated tube 110 from passing out of the stomach 102 and into the esophagus 106 against normal digestive flow. It should also be noted that the positioning member 158 can prevent the elongated tube 110 from forming kinks due to bending and/or misalignment.

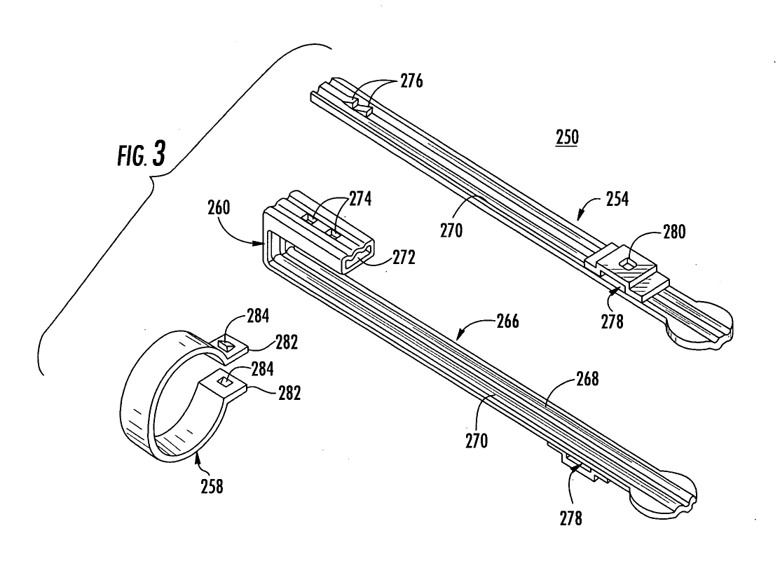

The clamping structure 152 can include a front side member 154 and a backside member (see FIG. 3) that extend along the length of stomach 102. The positioning member 158 can be coupled to the front side member 154 and the backside member. In addition to securely supporting the elongated tube 110, the positioning member 158 can also position the clamping structure 152 at a particular distance from one side of the stomach 102 to at least partially determine the capacity of the stomach 102 and the elongated tube 110. For instance, a relatively short positioning member 158 can position the clamping structure 152 relatively close to one side of the stomach 102 resulting in a stomach digestive region of relatively lesser capacity. Alternatively, a relatively long positioning member 158 can position the clamping structure 152 relatively far from one side of the stomach 102, resulting in a stomach digestive region of relatively greater capacity. Accordingly, the length of positioning member 158 can determine the position of the clamping structure 152.

In one arrangement, the positioning member 158 can be a ring adapter. In addition to the positioning member 158 having a shape that corresponds to the shape of the collar 113, the positioning member 158 can include curved portions designed to generally hug the curvature of the stomach 102. As noted above, positioning member 158 can be constructed of any suitable rigid, semi-rigid, or flexible material, such a plastic and/or rubber. A flexible positioning member can allow an increased range of movement, and thereby, providing an increased comfort level to the patient. A non-exhaustive list of the materials out of which a suitable flexible positioning member 158 can be constructed include flexible plastics and elastic materials. When assembled, the curved portion of the positioning member 158 can abut the stomach 102 and can couple to the front side member 154 and backside member of clamping structure 152. In this arrangement, the positioning member 158 can securely position the clamping structure 152 to the appropriate position on the stomach 102.

In addition to the positioning member 158 positioning the clamping structure 152, the positioning member 158 can also be dimensioned to provide another clamping device. Such a clamping device does not simply secure the elongated tube 110, but also reduces the capacity of the stomach 102 and/or the elongated tube 110.

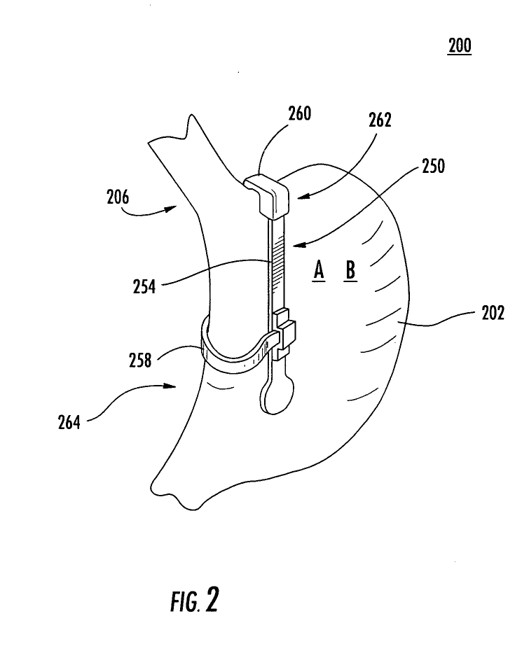

With the system 100 described noting the synergistic relationship amongst the components, the components of system 100 can be described in greater detail. Referring to FIGS. 2 and 3, a non-invasive stricture device 200 is illustrated in use with a stomach 202. The stricture device 200 is non-invasive because the stricture device 200 operates without any portion of the device piercing the stomach tissue.

Accordingly, the non-invasive stricture device 200 is positioned exterior to stomach 202 and does not penetrate the stomach 202. The stricture device 200 can include a front side member 254, a backside member 266, and a connecter 260 for coupling the front side member 254 to the backside member. Additionally, a positioning member 258 can be included for coupling to the front side member 254 and to the backside member 266 to form a clamping assembly. As discussed above, the positioning member 258 can also securely suspend an elongated tube within the interior of the stomach 102 and can function as a clamping means for establishing the appropriate capacity to reduce.

In the assembled arrangement, the front side member 254, the backside member 266, and the connector 260 can form a clamping structure 262 designed to have a size and shape suitable for fitting adjacent to the trachea side of the upper quadrant of a stomach 202 for regulating nutrient absorption and caloric intake by reducing the capacity of the stomach 202 and any regulating device within. The clamping structure 262 can be positioned adjacent to the cardiac orifice 206. In such an arrangement, the clamping structure 262 can restrict access to a substantial portion of the rest of the inside surface of the stomach 202. Nevertheless, it should also be noted that the device 200 can also be positioned so that the clamping structure 262 is orientated generally perpendicular to the esophagus (not shown). Further, it should be noted that the distance the clamping structure 262 is placed from the trachea can be individualized for individual patients and can be adjusted throughout the course of treatment. Accordingly, the clamping structure can be placed substantially at region A (as shown in FIG. 2), region B, and so forth.

The front side member 254 and the backside member 266 can be designed to any particular length, depending upon the physical dimensions of an individual patient and their need for treatment. Further, it should be noted that for some patients, the length ,of the front side member 254 can be different from the length of the backside member 266. Likewise, the dimensions of the connector 260 can also be customized to the individual patient's needs; however, it should be noted that the distance apart at which the connecter 260 sets the front side member 254 and the backside member 266 can determine the degree to which the stomach 202 is clamped. Accordingly, for a greater degree of clamping, the connecter 260 can set the front side member 254 and the backside member 266 relatively close. Alternatively, instead of the front side member 254 and the backside member 266 extending relatively parallel from the connector 260, the connector 260 can be designed so that the front side member 254 and the backside member 266 extend outward from the connector 260 at angles with one another. For instance, the front side member 254 and the backside member 266 can extend towards each other so that they are pre-biased and can provide a greater amount of clamping force.

The backside member 266 and the connector 260 can be an integral unit; however, it should be noted that the invention is not limited in this regard. For instance, the front side member 254 and the connector 260 can form an integral unit and the backside member 266, the front side member 254, and the connector 260 can form an integral unit. Nevertheless, the front side member 254, the backside member 266, and the connector 260 can each be individual pieces that can be assembled to form a clamping structure 262.

The skin contacting surfaces 268 of the front side member 254 and the backside member 266 can be free of piercing projections. As the clamping structure 262, and generally the device 200, can remain in a clamped arrangement at a particular position about the stomach 202, there is no need for such projections to anchor the device 200 or to maintain a clamped position. As a further benefit, with no attachment to the stomach 202, the front side member 254 and the backside member 266 can be removed for complete removal of device 200. Although the skin contacting surfaces 268 can be smooth to minimize trauma to the stomach 202 tissue, the skin contacting surfaces 268 can also include structure for allowing for fluid flow to bath the tissue. For example, such structure can include ridges 270 that allow for bathing of the tissue with beneficial bodily fluids by providing channels for fluid flow, encourage improved blood flow, and help prevent necrosis. Such structure can also include openings and apertures for allowing bathing of the tissue.

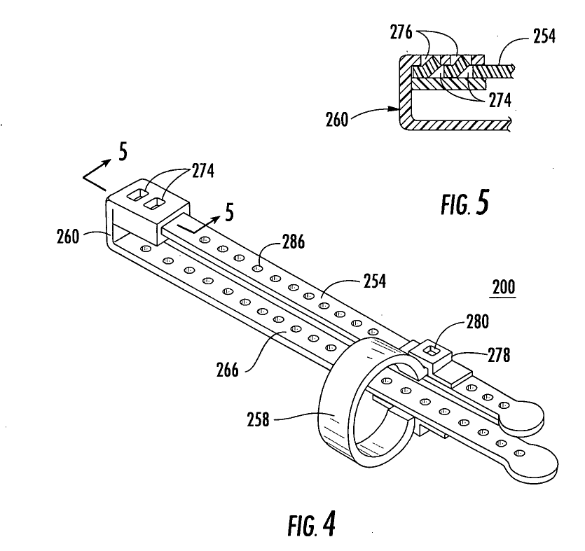

The connector 260 can include a slot 272 adapted to receive an end of the front side member 254 and/or the backside member 266. The slot 272 can be dimensioned so that the end of the front side member 254 and/or the backside member 266 fits snuggly and securely in the slot 272. Also, the connector 260 and the slot 272 of the backside member 266 and the front side member 254 can include corresponding engagement structure 276 for coupling the connector 260 to the front side member 254 and the backside member 266. In one arrangement of engagement structure 276, snap fitting members can be provided on the end of the front side member 254 and/or the backside member 266 for snap fitting in apertures 274 when an end is inserted into the slot 272 (as shown in FIG. 5 of a cross-section taken along line 5-5 of FIG. 4). Nevertheless, it should be noted that the invention is not limited in this regard as mechanical and/or adhesive attachments are also appropriate.

Similarly, the front side member 254 and the backside member 266 can include an anchoring slot 278 having an aperture 280. The anchoring slot 278 can be adapted to receive the end 282 of the positioning member 258. During insertion of end 282, snap fitting members 284 can snap engage the apertures 280 to securely couple the positioning member 258 to the front side member 254 and the backside member 266. Nevertheless, it should be noted that the invention is not limited in this regard as mechanical and/or adhesive attachments are also appropriate.

Further, the components of the non-invasive stricture device 200 can include rounded and curved portions so that corners and edges are not formed. For instance, a portion of the front side member 254 and the backside member 266 can include curved ends without sharp or angled portions. The curved and rounded portions can provide for smooth positioning and seating adjustments that may occur due to natural body movements.

The front side member 254, the connector 260, the backside member 266, and the positioning member 258, can be made of biologically inert materials, such as surgical steel or magnesium. Further, each individual component can be made of a different material compared to the other components.

In another embodiment of the non-invasive stricture device 200 shown in FIGS. 4 and 5, the front side member 254 and the backside member 266 can include a plurality of apertures 286. The apertures 286 can allow body fluids to contact the stomach 202. Also, the apertures 286 can provide suturing or anchoring positions. In such an embodiment, the front side member 254 and the backside member 266 can be sutured to the stomach 202 through apertures 286. For instance, such capabilities may help prevent unwanted movement when the positioning member 258 is not coupled to the front side member 254 and the backside member 266.

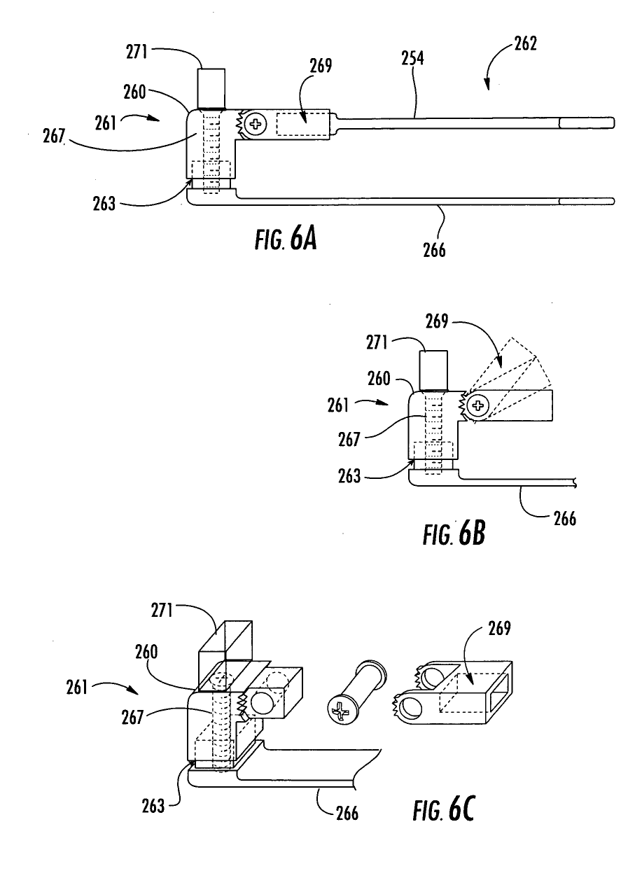

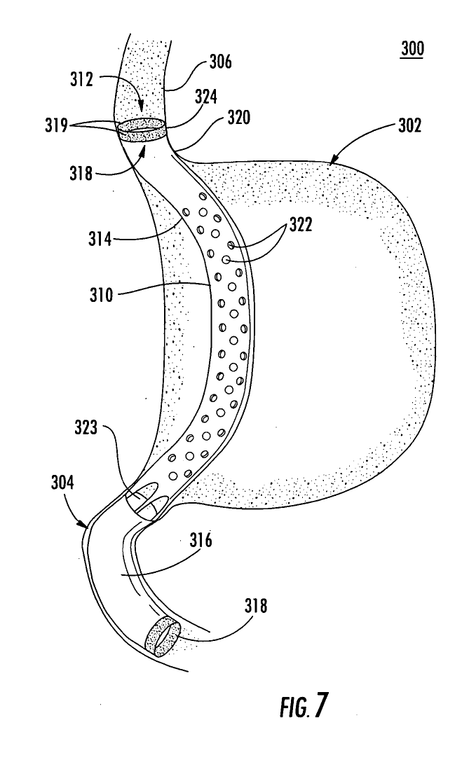

There is shown in FIGS. 6A, 6B, and 6C partial cross sections of another embodiment of a stomach stricture device 200 having a clamp adjusting mechanism for adjusting the clamping structure 262 as the dietary needs of the patient changes. The clamp adjusting mechanism allows the level of force provided by the clamping structure 262 to be adjusted throughout the treatment of the patient. The clamp adjusting mechanism can be any mechanism or arrangement of structures that can be used to adjust the distance between the front side member 254 and the backside member 266, and thereby, adjust the level of clamping force provided by the clamping structure 262.

The clamp adjusting mechanism can be as a motor screw assembly 261. The front side member 254 can be coupled to the connector 260 and the backside member 266 can include a portion that can enter a recess 263 in the connecter 260. A threaded structure 267 can extend through a portion of the connector 260 and can couple the backside member 266 to the connector 260. In this arrangement, rotation of the threaded structure 267 can adjust the distance between the front side member 254 and the backside member 266. It should be noted that the clamp adjusting mechanism is not limited to use with this particular embodiment of the stomach stricture device 200 and can be used with any of the embodiments discussed herein.

It should also be noted that an actuator 271 can be included for motorized operation of the clamp adjusting mechanism. The actuator 271 can be any appropriate device and/or systems for increasing and decreasing the amount of clamping force provided. For instance, a remote telemetry system can be used to send and receive signals from the actuator 271. The telemetry system can be used to instruct the actuator 271 to operate the clamp adjusting mechanism. In such a system, the signals can be electromagnetic radiation, such as signals transmitted via radio frequency.

As further shown, the front side member 254 can engage a receiving portion 269 of the connector 260. The receiving portion 269 can be angularly movable with the connector 260; however, the receiving portion 269 can be locked into any suitable position. The ability to angularly move the front side member 254 while inserted in the receiving portion 269 can facilitate positioning during implantation. Further, the front side member 254 can be exchanged for different front side members as needed throughout treatment. It should be noted that the receiving portion 269 that is angularly movable with the connector 260 is not limited to use with this particular embodiment of the stomach stricture device 200 and can be used with any of the embodiments discussed herein.

According to another aspect of the invention, a method for positioning a non-invasive stricture device is provided. The method includes providing a non-invasive stricture device having a front side member, a backside member, and a connector. A positioning member can also be provided but is not required. To expose the stomach, traditional or laparoscopic surgery can be performed. The backside member can be guided through a small canal excised through the fatty tissue on the backside of the stomach. The backside member can be placed adjacent to the trachea side of the upper quadrant of the human stomach. The connector can be coupled to the backside member by inserting one end. of the backside member into a slot provided by the connector. Alternatively, the backside member and the connector can be one integral component and can be inserted together. The connector can be positioned generally at the top of the stomach. In assembled arrangement, the connector can rest atop the stomach adjacent to the esophagus being sure to avoid pinching the major stomach aortic vessel.

The front side member can then be guided through the thoracic cavity. One end of the front side member can be inserted into a free slot on the connector and secured by the corresponding engagement structure. A positioning member can be used to couple the front side member to the rear side member. Different sized positioning members can be used to vary the position of the clamping structure. Once implanted, the stricture device can clamp the stomach so as to limit the capacity of the stomach to absorb nutrients and generally digest consumed substances.

Also, it should be noted that the different components of the stricture device can be sutured to the stomach. Further, the stricture device could also achieve the desired effect by choosing a placement that would run perpendicular to the esophagus with the top section being placed on either side. Once implanted, the non-invasive stricture device can be removed by generally following the method in reverse. The removal procedure should be accomplished relatively easily without leaving any appreciable tissue damage even to the outer surface of the stomach.

There is shown in FIG. 6 a device 300 for regulating nutrient absorption and caloric intake that can be used with a stomach stricture device discussed above and can also be used independently. The device 300 can include an elongated tube 310 and anchoring structure 318 for anchoring the elongated tube 310 to adjacent tissue. The elongated tube 310 defines a passage 312 to guide ingested material through the stomach 302 and through a portion of the small intestine 304 and can include a stomach portion 314 and a lower intestine portion 316. As illustrated, the stomach portion 314 is for positioning substantially in the stomach 302 of a patient, and the lower intestine portion 316 is for positioning substantially in a portion of the small intestine 304 of the patient.

The anchoring structure 318 can be any suitable structure, and for example can include a web mesh capable of supporting the elongated tube 310 when sutured to adjacent tissue. The web can include a reinforced region that can prevent tearing of the elongated tube 310 at the suture site. Although it is preferred that both the stomach portion 314 and the lower intestine portion 316 include anchoring structure 318, such an arrangement is not necessary, particularly in the arrangement where the device 300 is used in conjunction with system 100 discussed above.

In one embodiment, the stomach portion 314 can include an esophageal portion 320 for positioning substantially at the region where the esophagus 306 meets the stomach 302. This esophageal portion 320 can include the anchoring structure 318. Further, the anchoring structure 318 can be designed and shaped to provide a cardiac orifice structure 324 for anchoring the esophageal portion 320 to the adjacent tissue. In such an arrangement, substantially all ingested food passing into the stomach will be routed through passage 312.

The stomach portion 314 has one or more openings 322 for permitting ingested material to exit the stomach portion 314 into the stomach 302 and to enter the stomach portion 314 from the stomach 302. Similarly, the openings 322 can permit digestive agents, such as stomach acid, to enter the stomach portion 314 and to exit the stomach portion 314. It should be noted that it is preferred to have a plurality of such openings 322. These openings 322 can be of the same size, or different sizes. As the openings 322 facilitate the transfer of ingested material into and out of the stomach 302, and facilitate the transfer of digestive agents into and out of the elongated tube 310, the number and size of openings 322 can partially regulate nutrient absorption and caloric intake. For instance, if caloric intake and nutrient absorption should be drastically reduced for an obese patient, the elongated tube 310 can have limited number of openings 322 of relatively small diameters. Alternatively, if caloric intake and nutrient absorption should be only mildly reduced for a patient, the elongated tube 310 can have a greater number of openings 322 with relatively larger diameters. Additionally, elongated tube 310 can be constructed of a suitable selectively permeable and/or semi-permeable material that allows for the one directional and/or two directional exchange of ingested materials and digestive fluids.

In one aspect, the openings 322 can include exit openings 323 located at the junction between the stomach portion 314 and the lower intestine portion in the area of the pyloris 316. Although the exit openings 323 can be similar to the openings 322, the exit openings 323 are strategically located to allow any ingested material that has exited the elongated tube 310 to re-enter the tube 310 to avoid being trapped in the stomach 302. Additionally, it should be noted the exit openings 323 can be elongated in shape and/or generally greater in size than the openings 322 to facilitate the reentry of any sizeable quantity of ingested material. The openings 322 and the exit openings 323 can vary in size and shape depending on the individual patient and particular treatment needs and objectives.

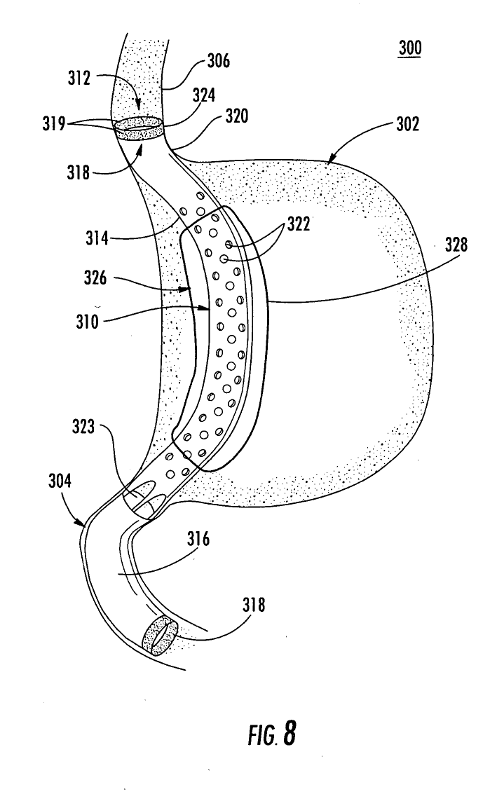

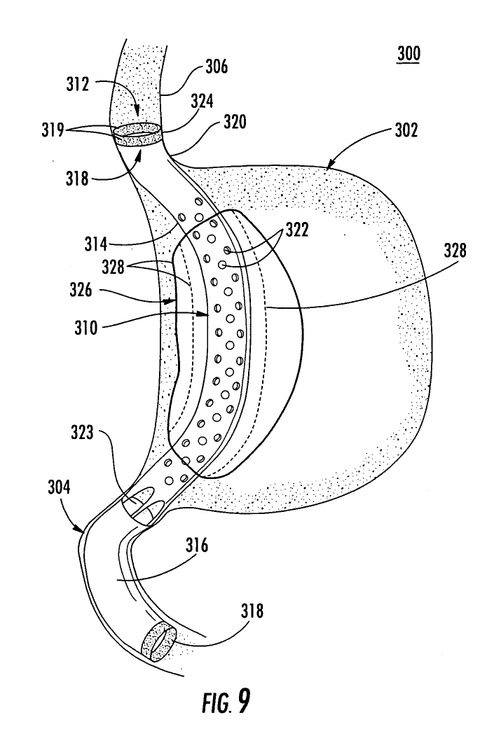

Alternative embodiments of the invention are shown in FIGS. 7-10. In these embodiments, the device 300 includes structure 326 for positioning the elongated tube 310 within the stomach 302. Preferably, the structure 326 can position the elongated tube 310 in a substantially straight line extending from the entrance of the stomach to the exit of the stomach. Nevertheless, the invention is not limited in this regard as curves in elongated tubes 310 may be preferable for some patients. The structure 326 can include any structure that extends from the elongated tube 310 to partially fill the interior cavity of the stomach 302. Accordingly, the structure 326 for positioning can not only position the elongated tube 310 appropriately, but can also prevent the elongated tube 310 from being removed from the stomach 302, in addition to providing the patient with a “full” stomach sensation.

In one example, the structure 326 can include an inflatable member 328 that extends outwardly from the elongated tube 310. The inflation member 328 can be attached to the stomach portion 314 and can be dimensioned to extend along almost the entire stomach portion 314, or can be limited to smaller dimensions. Alternatively, the inflation members 328 can be limited to circumferentially expanding from the elongated tube 310 without extending along the length of the tube 310. The inflation members 328 can be made of an elastic material that can allow movements associated with natural peristalsis. Alternatively, the inflation members 328 can be made of an inelastic but flexible sack of material. Furthermore, the inflation members can be inflated with gas, liquid, plasma, or a combination thereof. The inflatable members 328 can be inflated after insertion; however, the invention is not limited in this regard as the inflatable members 328 can be inflated before and/or during insertion.

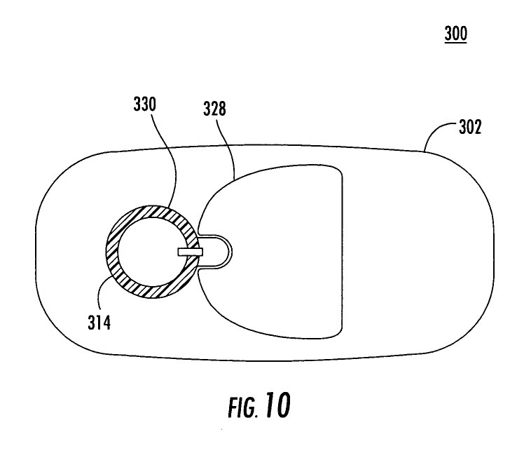

There is shown in FIG. 9 an embodiment with multiple inflation members 328. The inflation members 328 extend outwardly from the elongated tube 310 and generally travel the length of the stomach. Nevertheless, the invention is not limited in this regard as the inflation members 328 can be provided with various sizes and shapes and can be spaced variously along the elongated tube 310.

FIG. 10 is a cross-sectional view of one embodiment of device 300. In this cross-section view, the inflation member 328 is illustrated as being located on only one portion of the stomach portion 314. In such an arrangement, the inflation member 328 can ensure that the elongated tube extends on along a straight line from the entrance to the stomach to the exit of the stomach. Also, the it should be noted that channels 330 can extend along the elongated tube 310 for inflating the inflation members 328. Accordingly, with the channels 330, the inflation members 328 can be inflated after the stomach portion 314 is inserted into the stomach 302. The inflation members 328 can be inflated by inserting a small inflation tube down the patient's esophagus and connecting with the channels 330. Valves (not shown) can be included for preventing deflation.

Also in accordance with the inventive arrangements, a method for implanting a device for regulating nutrient absorption and caloric intake is provided. The method can include providing a device for regulating nutrient absorption and caloric intake having an elongated tube with a stomach portion and a lower intestine portion. The elongated tube can defining a passage to guide ingested material through the stomach and through a portion of the small intestine. Further, the device can include anchoring structure at the stomach portion for anchoring the elongated tube before the elongated tube enters the stomach. The device can be inserted through the throat of the patient using a variety of suitable tools, such as suitable laparoscopes and dispensing apparatuses. The device can be positioned with the stomach portion of the device substantially in the stomach of the patient and the lower intestine portion substantially in the lower intestine of the patient. The device can be secured to the tissue of the patient's digestive system with various means. In one example, the device can be sutured to the adjacent tissue at appropriate sections along the stomach and/or intestine. It should be noted that the device can be secured along multiple points of the device including the stomach portion and the lower intestine portion. Once inserted, a portion of the ingested material will pass through the passage without being digested by the patient.

The method can also include the step of adjusting the length of the lower intestine portion where a longer length prevents absorption of ingested material through the lower intestine. Further, inflation members can be provided. Inflation members can be secured to the stomach portion of the device and can be filed with one or more of a fluid, a gas, and plasma. The inflation members can be inflated with tubes inserted in the throat of the patient and connected to valves and or directly to channels provided by the inflation members. The inflation members can be adjusted after implantation is complete.

Also in accordance with the inventive arrangements, a method of regulating nutrient absorption and caloric intake is provided. Although the method can involve the use of the devices and systems discussed above, the method is not limited in this regard. The method can include the step of non-invasively reducing the capacity of the stomach. For example, a clamping device can be used to section a portion of the stomach into functioning and non-function sections. The clamping device can be one of the embodiments of stricture devices discussed above; however, the invention is not limited in this regard.

Further, it should be noted that the method also contemplates prolonged treatments where the dietary needs of the patient can change over time. Accordingly, the portion of clamped stomach that is considered as functional can be adjusted by re-positioning the clamping device. The clamping device can be re-positioned in any suitable manner, such as physically moving the clamping device, interchanging a different structure of the clamping device to affect the arrangement of the clamping device, and adjusting the clamping device having a clamp adjusting mechanism or other suitable adjustment means. Additionally, the level of clamping can also be selectively adjusted, such as incrementally over time as treatment progresses.

The method can also include the step of limiting the interaction of digestive agents with ingested substances. The interaction of digestive agents with ingested materials can be accomplished in two or more manners. The ingested material can be funneled through the digestive system. Funneling prevents the ingested material from mixing with the digestive agents that are naturally in the digestive system. Nevertheless, limiting the interaction also includes preventing and/or limiting the amount of digestive agents that enter the funnel. For instance, openings can be provided in the funnel. The positioning, size, and amount of the openings can all be varied to regulate this interaction. Generally, a greater number of openings with a greater opening area allow for an increased level of nutrients absorption and caloric intake.

Additionally, it should be noted that the method can include only some of the steps listed above and can include others not discussed. Also, as no permanent changes are made to the patients anatomy, the method can be stopped and the patient can return to the normal digestive process at any time.

This invention can be embodied in other forms without departing from the spirit or essential attributes thereof. Accordingly, reference should be made to the following claims, rather than to the foregoing specification, as indicating the scope of the invention.

1. A system for regulating nutrient absorption and caloric intake, comprising:
an elongated tube having a stomach portion and a lower intestine portion, the elongated tube defining a passage to guide ingested material through the stomach and through a portion of the small intestine;
a non-invasive stomach stricture device having a positing member; and
wherein the positioning member positions the elongated tube within the stomach.

2. The system according to claim 1, wherein the non-invasive stomach stricture device further includes a clamping structure for regulating the rate of flow of ingested material through the elongated tube;
wherein the clamping structure alters the capacity of the portion of stomach through which the elongated tube passes.

3. The system according to claim 1, the elongated tube further comprising an inflatable member.

4. The system according to claim 1, wherein the clamping structure prevents the elongated tube from passing out of the stomach.

5. The system according to claim 1, wherein the positioning member positions the clamping structure on the stomach and relative to the elongated tube.

6. The system according to claim 1, wherein the clamping structure includes a front side member and a backside member, and a connector for coupling to the front side member and the backside member.

7. The system according to claim 1, wherein the positioning member is dimensioned to clamp the elongated tube through the stomach and reduce the capacity of the passage.

8. The system according to claim 1, wherein the elongated tube includes a collar designed to be clamped by the positioning member, the positioning member is dimensioned and positioned on the outside of the stomach to clamp the collar for suspending a portion of the elongated tube within the stomach.

9. The system according to claim 1, wherein the positioning member prevents the elongated tube from passing out of the stomach.

10. The system according to clam 1, wherein the non-invasive stomach stricture device includes a clamp adjusting mechanism.

11. The system according to claim 10, wherein the clamp adjusting mechanism includes an actuator for operating the clamp adjusting mechanism.

12. A system for regulating nutrient absorption and caloric intake, comprising:
an elongated tube having a stomach portion and a lower intestine portion, the elongated tube defining a passage to guide ingested material through the stomach and through a portion of the small intestine;
a non-invasive stomach stricture device having a clamping structure; and
wherein the clamping structure prevents the elongated tube from exiting the stomach.

13. The system according to claim 12, wherein the non-invasive stomach stricture device includes a positioning member for positioning the elongated tube within the stomach.

14. The system according to claim 12, wherein the arrangement of the clamping structure regulates the rate of flow of ingested material through the elongated tube.

15. The system according to claim 12, wherein the arrangement of the clamping structure alters the capacity of the portion of stomach through which the elongated tube passes.

16. The system according to claim 12, the elongated tube further comprising an inflatable member.

17. The system according to claim 12, wherein the clamping structure includes a front side member, a backside member, and a connector for coupling to the front side member and the backside member.

18. The system according to claim 17, wherein the front side member, the backside member, and the connector are integral.

19. The system according to claim 12, wherein the elongated tube includes a collar designed to be clamped by the positioning member, the positioning member is dimensioned to clamp the collar for suspending a portion of the elongated tube within the stomach.

20. The system according to clam 12, wherein the non-invasive stomach stricture device includes a clamp adjusting mechanism.

21. The system according to claim 20, wherein the clamp adjusting mechanism includes an actuator for operating the clamp adjusting mechanism.

22. A non-invasive stomach stricture device, comprising:
a front side member;
a backside member;
a connector for coupling the front side member to the backside member to form a clamp, wherein the clamp is designed to clamp a stomach.

23. The device according to claim 22, wherein the clamp is designed to have a size and shape suitable for fitting adjacent to the trachea side of the upper quadrant of a stomach for regulating nutrient absorption and caloric intake by reducing the capacity of the stomach.

24. The device according to claim 22, wherein the backside member and the front side member include stomach contacting surfaces, the stomach contacting surfaces being free of piercing projections.

25. The device according to claim 24, wherein at least one of the stomach contacting surfaces include structure for permitting fluid flow.

26. The device according to claim 22, further comprising:
a positioning member for coupling to the front side member and the backside member to form a clamping assembly.

27. The device according to claim 22, wherein the front side member, connector, and backside member are integral.

28. The device according to claim 22, wherein the backside member and the connector are integral.

29. The device according to claim 22, wherein the connector comprises a slot adapted to receive the front side member so that the front side member can be coupled to the backside member to form a clamp.

30. The device. according to claim 26, wherein the backside member and the front side member each comprise an anchoring slot, each anchoring slot adapted to receive an end of the positioning member.

31. The device according to claim 30, wherein each slot and the positioning member include corresponding engagement structure for coupling the positioning member to the front side member and the backside member.

32. The device according to claim 22, wherein the front side member and the backside member include an aperture through which the front side member and the backside member can be sutured to the stomach.

33. A non-invasive stomach stricture device, comprising:
a front side member;
a backside member;
a connector connecting the front side member to the backside member, the connector dimensioned to space the front side member from the backside member such that the device can be positioned over the stomach of a patient with the front side member over a front side of the stomach and the backside member positioned over the backside of the stomach, and the stomach will be constricted between the front side member and the backside member.

34. The device according to claim 33, wherein the connector and the backside member are integral.

35. The device according to claim 33, wherein the connector determines the distance between the front side member and the backside member.

36. The device according to claim 33, further comprising a positioning member for positioning the stomach stricture device on the stomach of a patient.

37. The device according to claim 36, wherein the positioning member couples to a portion of the front side member and to a portion of the backside member.

38. The device according to claim 36, wherein the positioning member is adjustable relative to the front side member and the backside member.

39. The device according to claim 37, wherein the backside member and the front side member each comprise an anchoring slot for receiving an end of the positioning member.

40. The device according to claim 39, wherein the positioning member comprises engagement structure for connecting with at least one corresponding engagement structure in at least one of the front side member and the backside member.

41. The device according to claim 33, wherein the front side member is separable from the backside member for allowing the device to be removed from a patient.

42. The device according to claim 33, wherein the device is dimensioned for positioning adjacent to the trachea side of the upper quadrant of a human stomach to limit the rate of flow of ingested material into the stomach and to limit the digestion and absorption of the ingested material.

43. The device according to claim 33, wherein the device is dimensioned for positioning adjacent to the upper quadrant of a human stomach, substantially perpendicular to the esophagus, to limit the rate of flow of ingested material into the stomach and to limit the digestion and absorption of the ingested material.

44. A method for implanting a non-invasive stomach stricture device, comprising the steps of:
providing a non-invasive stomach stricture device having a front side member, a backside member, and a connector for coupling the front side member to the backside member to form a clamp, wherein the clamp is designed to clamp a stomach;
coupling the backside member to the connector;
positioning the backside member, coupled to the connector, adjacent to the trachea side of the upper quadrant of the human stomach; and
coupling the front side member to the connector to form a clamp which constricts the stomach to limit the rate of flow of ingested material into the stomach and to limit the amount of usable inside surface area of the stomach.

45. The method according to claim 44, wherein the step of placing the backside member adjacent to the trachea side of the upper quadrant of the human stomach comprises excising a canal through fatty tissue on the backside of the stomach.

46. The method according to claim 44, further comprising the step of connecting a connector to the front side member and the backside member.

47. A device for regulating nutrient absorption and caloric intake, comprising:
an elongated tube having a stomach portion and a lower intestine portion, the elongated tube defining a passage to guide ingested material through the stomach and through a portion of the small intestine; and
an anchoring structure for anchoring the elongated tube to adjacent tissue.

48. The device according to claim 47, wherein the stomach portion includes an esophageal portion for positioning substantially at the region where the esophagus meets the stomach, the esophageal portion including the anchoring structure.

49. The device according to claim 47, wherein the anchoring structure includes at least one of a cardiac orifice structure for anchoring a portion of the elongated tube to the cardiac orifice, a collar, a web, a and reinforced portion.

50. The device according to claim 47, further comprising anchoring structure at the lower intestine portion for anchoring the lower intestine portion to adjacent tissue of the patient.

51. The device according to claim 50, wherein the anchoring structure includes at least one of a collar, a web, and a reinforced portion.

52. The device according to claim 47, wherein the stomach portion includes at least one opening.

53. The device according to claim 47, further comprising structure for positioning the tube in the stomach of a patient.

54. The device according to claim 53, wherein the positioning structure comprises at least one inflation member.

55. The device according to claim 46, further comprising a plurality of inlet openings located substantially at the region between the stomach portion and the lower intestine portion.

56. The device according to claim 55, wherein the inlet openings are elongated slots.

57. The device according to claim 46, further comprising a plurality of exit openings located substantially at the region between anchoring structure an the stomach portion.

58. A method for regulating nutrient absorption and caloric intake, comprising the steps of:
providing a device for regulating nutrient absorption and caloric intake having an elongated tube with a stomach portion and a lower intestine portion, the elongated tube defining a passage to guide ingested material through the stomach and through a portion of the small intestine, and an anchoring structure at the stomach portion for anchoring the elongated tube before the elongated tube enters the stomach;
positioning the device in a patient with the stomach portion substantially in the stomach of the patient and the lower intestine portion substantially in the lower intestine of the patient;
securing the device in position in the patient;
whereby a portion of the ingested material will pass through the passage without being digested by the patient.

59. The method of claim 58, further comprising the step of adjusting the length of the lower intestine portion, wherein a longer length prevents absorption of ingested material through the lower intestine.

60. The method of claim 58, further comprising the step of providing inflation members secured to the stomach portion, and filling the inflation members after the stomach portion has been positioned in the stomach of the patient.

61. The method of claim 58, further comprising the step of securing the lower intestine portion to the lower intestine of the patient.

62. A method of regulating nutrient absorption and caloric intake, comprising the steps of:
non-invasively reducing the capacity of the stomach; and
limiting the interaction of digestive agents with ingested substances.

63. The method of claim 62, wherein reducing the capacity of the stomach includes clamping a portion of the stomach.

64. The method of claim 63, further comprising the step of selectively adjusting the clamping force.

65. The method of claim 62, wherein the limiting step includes funneling ingested material through a portion of the digestive system.
